SEQUENCE LISTING (10 sequences)

SEQ ID NO: 1 - protein, 19 aa, Artificial Sequence
  synthetic construct
TRGVEEVAAIPEVLRRRGN

SEQ ID NO: 2 - protein, 4 aa, Artificial Sequence
  synthetic construct
RRRG

SEQ ID NO: 3 - protein, 273 aa, Artificial Sequence
  synthetic construct
SLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLGGGSGGGSTRKHKILHRLLQDSSRRRGN

SEQ ID NO: 4 - protein, 13 aa, Artificial Sequence
  synthetic construct
KHKILHRLLQDSS

SEQ ID NO: 5 - protein, 5 aa, Artificial Sequence
  synthetic construct
RRRGN

SEQ ID NO: 6 - protein, 5 aa, Artificial Sequence
  synthetic construct
YALAA

SEQ ID NO: 7 - protein, 245 aa, Homo sapiens
SLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRL

SEQ ID NO: 8 - protein, 8 aa, Artificial Sequence
  synthetic construct
GGGSGGGS

SEQ ID NO: 9 - protein, 11 aa, Artificial Sequence
  synthetic construct
KHKILHRLLQD

SEQ ID NO: 10 - protein, 9 aa, Homo sapiens
YPYDVPDYA

(19) United States
(12) Patent Application Publication
(10) Pub. No.: US 2014/0255361 A1
(43) Pub. Date: Sep. 11, 2014

(54) ESTROGEN-RECEPTOR BASED LIGAND SYSTEM FOR REGULATING PROTEIN STABILITY

(71) Applicant: The Board of Trustees Of The Leland Stanford Junior University, Stanford, CA (US)

(72) Inventors: Thomas J. Wandless, Menlo Park, CA (US); Ling-chun Chen, Fremont, CA (US)

(73) Assignee: The Board of Trustees Of The Leland Stanford Junior University, Stanford, CA (US)

(21) Appl. No.: 14/201,447

(22) Filed: Mar. 7, 2014

Related U.S. Application Data
(60) Provisional application No. 61/774,524, filed on Mar. 7, 2013.

(51) Int. Cl. C07K 14/005; C07K 7/08; A61K 38/16

(52) U.S. Cl. CPC C07K 14/005; A61K 38/162; C07K 7/08
USPC 424/93.21; 514/44 R; 530/326; 530/350; 435/69.7; 435/325

(57) ABSTRACT

Disclosed herein are systems, methods and compositions for rapidly and reversibly destabilizing a target protein in vitro or in vivo, in the presence or absence of a cell-permeable, synthetic molecule or ligand.

### CROSS-REFERENCE TO RELATED APPLICATIONS

This application claims the benefit of U.S. Provisional Application No. 61/774,524, filed Mar. 7, 2013, incorporated herein by reference in its entirety.

### STATEMENT REGARDING GOVERNMENT INTEREST

This invention made with Government support under contract GM073046 awarded by the National Institutes of Health. The Government has certain rights in this invention.

### REFERENCE TO SEQUENCE LISTING, TABLE OR COMPUTER PROGRAM

A Sequence Listing is being submitted electronically via EFS in the form of a text file, created Mar. 7, 2014, and named “091511-0583_ST25.txt” (6,376 bytes), the contents of which are incorporated herein by reference in their entirety.

### TECHNICAL FIELD

Compositions, systems and methods for rapidly and reversibly destabilizing a target protein in vitro or in vivo, using cell-permeable, synthetic molecules are described. The coding sequence for a protein of interest is genetically fused to a sequence encoding a stability-affecting protein domain capable of interacting with a small-molecule ligand, and the presence and/or amount of ligand conditionally induces degradation of the fusion protein.

### BACKGROUND

Techniques for modulating transcription of DNA and RNA expression provide powerful tools for studying specific genes and their biological function. For example, the tet/dox and Cre/lox systems have been widely used to target gene expression at the transcriptional level (Ryding, A. D. S. et al. (2001) J. Endocrinol. 171:1-14), and RNA interference provides a method to achieve post-transcriptional gene silencing (Fire, A. et al. (1998) Nature 391:806-811; Medema, R. H. (2004) Biochem. J. 380:593-603; Raab, R. M. and Stephanopoulos, G. (2004) Biotechnology & Bioengineering 88:121-132). Biological studies of mammalian development and physiology have also been greatly aided by techniques allowing the disruption of specific genes using homologous recombination and generation of transgenic mice. However, interpretation of the phenotypes of transgenic or knock-out mice possessing null mutations can be hampered by early embryonic lethality or compensation for the absence of a gene during development. Toward a solution for such problems, methods for conditional gene inactivation have been developed, but often these methods are slow and irreversible. There remains a widespread need for a facile method to reversibly inactivate the protein product of a specific gene rather than permanently knocking-out the gene encoding the protein.

Techniques have also been developed to regulate proteins on a post-translational level. Experimental methods have been developed to regulate protein stability and function rapidly and reversibly using protein domains that are conditionally stable in cultured cells or living animals. Such methods are often controlled by the binding of a small molecule ligand. (Baker, M. Nat. Methods 9, 443-447 (2012)). Methods to conditionally regulate protein abundance in cells are useful for biologists to study a protein's function(s) in complex biological systems. However, methods for regulating protein function directly are limited, especially in mammalian cells. Inhibitors or activators of particular proteins have been identified, and often take the form of cell-permeable small molecules. Many of these molecules have found widespread use as biological probes, often because the speed, dosage-dependence, and reversibility of their activities, which complement methods for genetically modulating gene expression (Schreiber, S. L. (2003) Chem. & Eng. News 81:51-61). However these inhibitors or activators are often promiscuous, affecting several proteins rather than a specific protein (Davies, S. P. et al. (2000) Biochem. J. 351:95-105; Bain, J. et al. (2003) Biochem. J. 371:199-204; Godl, K. et al. (2003) Proc. Natl. Acad. Sci. U.S.A. 100:15434-15439; Tan, D. S. (2005) Nat. Chem. Biol. 1:74-84; Mayer, T. U. et al. (1999) Science, 286:971-974).

A method by which specific kinases can be inhibited using a small-molecule modulator has also been developed (Shah et al., 1997; Bishop, A. C. et al. (1998) Current Biology 8:257-266). This method involves mutating the protein of interest, typically replacing a large conserved residue in the active site with a smaller residue, such as glycine or alanine. Specificity is achieved by chemically modifying a promiscuous inhibitor to include a bulky side-chain substituent (e.g. R-group), which fills the corresponding cavity in the binding site of the modified protein of interest, while preventing productive interactions with other kinases. While this so-called “bump-hole” approach has been successful both in cultured cells and in mice (Bishop, A. C. et al. (2000) Nature 407:395-401; Wang, H. et al. (2003) Proc. Natl. Acad. Sci. U.S.A. 100:4287-4292, Chen, X. et al. (2005) Neuron 46:13-21), it appears to be limited to ATPases and GTPases. Additional methods are required to probe the function of a wider variety of proteins.

Alternative strategies to perturb protein function by exploiting existing cellular processes have also been devised (Banaszynski, L. A. et al. (2006) Chem. Biol. 13:11-21). For example, a method has been developed for controlling protein function based on the importance of certain N-terminal residues for protein stability (Bachmair, A. et al. (1986). Science 234:179-186). Szostak and coworkers showed that a small peptide sequence could be fused to the N-terminus of a protein of interest to modulate protein stability (Park, E-C. et al. (1992) Proc. Natl. Acad. Sci. U.S.A. 89:1249-1252). Varshavsky and coworkers have further isolated a temperature-sensitive peptide sequence that greatly reduced the half-life or dihydrofolate reductase (DHFR) at the non-permissive temperatures (Dohmen, R. J. et al. (1994) Science 263:1273-1276). This approach has been used to study proteins in yeast (Labib, K. et al. (2000) Science 288:1643-1646; Kanemaki, M. et al. (2003) Nature 423:720-724). Furthermore, dimeric small molecules have been engineered to conditionally target fusion proteins for degradation via E3 ligase or the proteasome, itself (Schneekloth et al., 2004; Janse, D. M. et al. (2004) J. Biol. Chem. 279:21415-21420). However, these systems require either a prior knowledge of the high-affinity ligands that modulate the activity of a protein of interest or they are restricted to genetically engineered yeast strains.

An alternative approach for controlling protein function directly is to interfere with subcellular localization. For example, several methods have been developed to regulate protein localization using a small-molecule by taking advantage of the FKBP-rapamycin-FRB ternary complex (Kohler, J. J. et al. (2003) Chem. Biol. 10:1303-1331 and Inoue, T. et al. (2005) Nature Methods 2:415-418). Rapamycin and FK506 are potent, commercially available immunosuppressive agents, which are ligands of the FK506-binding protein (FKBP12, FKBP). Rapamycin also binds to FKBP-rapamycin-associated protein (FRAP). FRAP is also called the mammalian target of rapamycin (mTOR), rapamycin and FKBP target 1 (RAFT1), and FKBP-rapamycin-binding (FRB). Rapamycin binds to and inhibits FRAP/mTOR by interacting with its FRB domain to inhibit/delay G1 cell cycle progression in mammalian cells (see, e.g. Choi, J. et al. (1996) Science 273:239-42 and Vilella-Bach, M. et al. (1999) J. Biol. Chem. 274:4266-72. The FRB domain is required for FKBP-rapamycin-associated protein kinase activity and G1 progression. Fusions of proteins of interest can be made to either FKBP or to the FRP domain of FRAP/mTOR. Colocalization of the protein of interest is induced upon addition of rapamycin. Because rapamycin has inherent biological activity, researchers have developed a “bump-hole” strategy (similar to that described above), wherein rapamycin derivatives possessing large substituents at the FRB binding interface bind poorly to the wild-type FRB domain and thus the target FRAP/mTOR; binding is restored upon introduction of compensatory cavity-forming mutations in FRB. Specifically, a C20-methallyl-rapamycin derivative (MaRap) binds to a triple-mutated variant of FRB called FRB* (Liberles, S. D. et al. (1997) Proc. Natl. Acad. Sci. U.S.A. 94:7825-7830).

Recently, numerous applications have been described employing light responsive protein domains. The LOV (Light-Oxygen-Voltage) domains are part of plant photoreceptor proteins and detect blue light via a flavin cofactor. (Herrou, J. et al. Nat. Rev. Microbiol. 9, 713-723 (2011)). The LOV2 domain of oat phototropin 1 (AsLOV2) holds a c-terminal alpha helix that is tightly bound to the LOV core domain in dark. Exposure to light results in unfolding of the helix. The AsLOV2 domain has been used to regulate certain protein activities by the steric inhibition of an effector protein or by restricting a specific protein conformation. (Wu, Y. I. et al. Nature 461, 104-108 (2009); Lee, J. et al. Science 322, 438-442 (2008); Strickland, D., et al. Proc. Natl. Acad. Sci. USA 105, 10709-10714 (2008)). These methods are suitable for the reported engineered proteins but not generally applicable to other proteins. Alternatively, engineered AsLOV2 and other photosensory domains have been used to establish light mediated protein-protein interactions. (Shimizu-Sato, S., et al. Nat. Biotechnol. 20, 1041-1044 (2002); Levskaya, A., et al. Nature 461, 997-1001 (2009); Yazawa, M., et al. Nat. Biotechnol. 27, 941-945 (2009); Kennedy, M. J. et al. Nat. Methods 7, 973-975 (2010); Strickland, D. et al. Nat. Methods 9, 379-384 (2012); Wang, X., et al. Nat. Methods 9, 266-269 (2012); Polstein, L. R. et al. J. Am. Chem. Soc. 134, 16480-16483 (2012); Lungu, O. I., et al. Chem. Biol. 19, 507-517 (2012); Zhou, X. X. et al. Science 338, 810-814 (2012)). A small four amino acid peptide degron, RRRG (SEQ ID NO: 2), has also been reported (Bonger, K. M., et al. Nat. Chem. Biol. 7, 531-537 (2011)), and it was shown that a C-terminal fusion of this peptide to a protein of interest results in rapid proteasome-mediated degradation in mammalian cells. Other degrons are known and recognized in bacteria (the YALAA peptide; hereinafter SEQ ID NO: 6) and yeast (the CL1 degron). Thus, translocation strategies to the cell membrane or the nucleus can allow location specific protein activity and light induced gene expression systems respectively.

These technologies may be useful in some cases, but they often lack the ability to control protein levels once present in the cells, and none of the existing methods developed so far is suitable for fast and reversible regulation of protein levels. Furthermore, while the aforementioned methods for regulating protein function directly are noteworthy, a need remains for a convenient, general method for regulating protein function, particularly a method that does not require the interaction of multiple proteins. Regulation of protein stability in cells in a more spacial or temporal manner is also desirable. Thus, a long-felt need remains for improved and novel systems and methods to regulate protein levels and functions directly, rapidly and reversibly using protein domains that are conditionally stable in cultured cells or living animals, particularly in mammalian/metazoan cells and mammalian organisms.

The foregoing examples of related art and limitations related thereto are intended to be illustrative and not exclusive. Other limitations of the related art will become apparent to those of skill in the art upon a reading of the specification and a study of the drawings.

### BRIEF SUMMARY

Compositions, systems and methods for modulating the stability of proteins in vitro and in vivo using cell-permeable small molecules are described. The coding sequence for a protein of interest is genetically fused to a sequence encoding a stability-affecting protein domain capable of interacting with a small-molecule ligand, the presence, absence, or amount of which is used to modulate the stability of the fusion protein.

In one aspect, a ligand-regulated conditional protein stability system is provided, comprising a nucleic acid sequence encoding a fusion protein that comprises a protein of interest fused in-frame to a sequence encoding a ligand-responsive, stability-affecting polypeptide domain, and a corresponding ligand which binds to the ligand-responsive, stability-affecting polypeptide domain, wherein, upon introduction of the nucleic acid sequence to a cell, the fusion protein is expressed, and the stability and/or level (quantity) of the fusion protein can be modulated by administering the ligand to the cells, tissue or organism. In some embodiments, the system employs a ligand-responsive, stability-affecting polypeptide domain based on the estrogen receptor (ER) and one or more ER-ligands (agonists or antagonists). In some embodiments, the ligand-responsive, stability-affecting polypeptide domain employed is the estrogen receptor ligand-binding domain (ERLBD). In some embodiments, the ER-ligand is selected from the group consisting of CMP8 (9a-(4-Chlorobenzyl)-7-hydroxy-4-[4-(2-piperidin-1-ylethoxy)phenyl]-1,2,9,9a-tetrahydro-3H-fluoren-3-one), 4-hydroxytamoxifen, fulvestrant and raloxifene.

The stability-affecting polypeptide domain can be used in the system disclosed herein to stabilize or destabilize a protein of interest (POI) depending on which ligand is used in the system. In some embodiments, the stability-affecting polypeptide domain acts as a degradation domain (DD)/ligand-induced stability domain, for example in the presence of estradiol as a ligand. In some embodiments, the stability-affecting polypeptide domain acts as a ligand-induced destabilization domain, for example in the presence of tamoxifen as a ligand.

In some embodiments, cells are transformed with the nucleic acid. In some embodiments, eukaryotic cells are transformed with the nucleic acid to produce a stably transformed eukaryotic cell. In some embodiments, the transformed cells are implanted into a living animal. In some embodiments, the transformed cells are implanted into immunodeficient mice as xenografts.

In some embodiments, the nucleic acid sequence is in a viral vector. In some embodiments, the viral vector is a pox virus. In some embodiments, the viral vector is a vaccinia virus.

In some embodiments, the protein of interest is a reporter protein. In some embodiments, the protein of interest is a therapeutic protein.

In some embodiments, the LID comprises SEQ ID NO: 1.

In some embodiments, the ligand-regulated, stability-affecting system comprises the amino acid sequence identified by SEQ ID NO: 3.

In some embodiments, the nucleic acid sequence is in a viral vector.

In some embodiments, the viral vector is a vaccinia virus.

In some embodiments, the ligand-responsive, stability-affecting polypeptide domain destabilizes the protein of interest or fusion protein in the presence of the corresponding ligand. In some embodiments, the ligand is CMP8 or 4-hydroxytamoxifen. In some embodiments, the ligand-responsive, stability-affecting polypeptide domain does not destabilize the protein of interest in the absence of ligand. In some embodiments, the ligand-responsive, stability-affecting polypeptide domain destabilizes the protein of interest or fusion protein in the presence of ligand to a greater degree or extent than it destabilizes the protein of interest or fusion protein in the absence of the ligand.

In some embodiments, the ligand-responsive, stability-affecting polypeptide destabilizes the protein of interest or fusion protein by causing an increase in the degradation or destruction of the protein of interest or fusion protein when exposed to ligand, as compared to the level of degradation of the protein of interest or fusion protein when not exposed to the ligand.

In some embodiments, the ligand-responsive, stability-affecting polypeptide changes the conformation of the fusion protein upon exposure to the corresponding ligand.

The stability-affecting polypeptide domain can be used in the system disclosed herein to stabilize or destabilize a protein of interest (POI) depending on which ligand is used in the system. In some embodiments, the stability-affecting polypeptide domain acts as a degradation domain (DD)/ligand-induced stability domain, for example in the presence of estradiol as a ligand. In some embodiments, the stability-affecting polypeptide domain acts as a ligand-induced destabilization domain, for example in the presence of tamoxifen as a ligand. In some aspects, a ligand-induced destabilizing/degradation domain (DD) is provided. In some embodiments, an improved FKBP12-based “ligand-induced destabilizing” (LID) domain is provided. In some embodiments, the LID is SEQ ID NO: 1. In some embodiments, an ERLBD-based LID system is provided.

In some embodiments, the system comprises (1) a fusion protein comprised or consisting or consisting essentially of a ligand-binding domain; an optional spacer; an optional 13-residue peptide (KHKILHRLLQD; herein identified as SEQ ID NO: 9) followed by 2 amino acids (SS) created from a restriction site; and a degron; and (2) a ligand. In some embodiments, a fusion protein is provided, wherein the fusion protein comprises SEQ ID NO: 3 or is a protein consisting of a sequence with at least about 80%, 85%, 90% or 95% sequence identity to SEQ ID NO: 3.

In some aspects, a method is provided for conditionally stabilizing a protein of interest, comprising fusing a nucleic acid encoding a protein of interest in-frame to a nucleic acid encoding a ligand-regulated, stability-affecting polypeptide to produce a nucleic acid encoding a fusion protein; introducing the nucleic acid encoding the fusion protein into a cell; expressing the fusion protein in the cell; and administering to the cell the corresponding ligand, to destabilize the fusion protein.

In some embodiments of the method, the cell is stably transformed with the nucleic acid. In some embodiments of the method, the stably transformed cell is implanted in a living animal. In some embodiments of the method, the nucleic acid sequence is in a viral vector. In some embodiments of the method, the viral vector is a vaccinia virus.

In some embodiments of the method, the ligand-responsive, stability-affecting polypeptide domain employs the estrogen receptor (ER) and one or more ER-ligands (agonists or antagonists). In some embodiments, the ER-ligand is selected from the group consisting of CMP8, 4-hydroxytamoxifen, fulvestrant and raloxifene.

In some embodiments of the method, the step of introducing the nucleic acid into a cell comprises infecting the cell with a viral vector comprising the nucleic acid, and implanting the cell into an animal. In some embodiments, the step of introducing the nucleic acid into a cell comprises transfecting a cell of a living animal, in which the cell resides.

In some aspects, provided is a cell comprising a nucleic acid encoding a fusion protein, wherein the fusion protein comprises a protein of interest fused in frame to a ligand-regulated, stability-affecting polypeptide.

In some aspects, provided is a kit of parts comprising a ligand-regulated conditional protein stability system, along with instructions for use.

Additional embodiments of the present methods and compositions and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B show systems for ligand-induced degradation of proteins, where FIG. 1A shows a prior art system composed of a destabilizing domain (DD) fused to a protein-of-interest (POI) in which the POI is degraded in the absence of ligand; addition of ligand to the system prevents degradation of the target protein. FIG. 1B shows an engineered “ligand-induced degradation” (LID) domain in accord with the present invention, where the LID is genetically fused to a POI, where expression of the fusion construct results in a stable fusion protein in the absence of ligand; the addition of a high-affinity ligand causes rapid and processive degradation of the LID domain and fusion protein.

FIG. 2 compares the stabilities of two LID domain fusion proteins.

FIG. 3 presents the stability of the GFP-tagged ERLID fusion protein in the presence of various small molecule ligands.

FIG. 4 shows the effects of several ligand antagonists, 4-hydroxytamoxifen (4-OHT), fulvestrant, and raloxifen on GFP-ERLID fusion protein stability in NIH3T3 cells.

FIG. 5 shows the reversibility of the stability-affecting polypeptide domain, where median fluorescence intensity of the fusion protein GFP-ERLIP expressed in NIH3T3 cells is shown in the presence of no ligand (no drug) as a control, after 4 hours exposure to estradiol (E2) or 4-hydroxytamoxifen (4-OHT), and then after removal of the E2 by washing (E2 wo) and after removal of E2 by washing with the addition of 4-hydroxytamoxifen (E2 wo+4OHT).

FIG. 6A-6D are Western blots that show the expression of stable fusion proteins GFP-ERLID (FIG. 6A), HA-Bax-ERLID (FIG. 6B), HA-hRPA2-ERLID (FIG. 6C) and HA-hUBC13-ERLID, and the destabilization of the fusion proteins in the presence of 4-hydroxytamoxifen (4-OHT) and stabilization of the fusion proteins in the presence of estradiol (E2). GAPDH serves as a leveling control and the antibodies were mouse anti-GFP antibody and rat anti-HA-antibody.

FIG. 7A shows the effects of several ligand antagonists, 4-hydroxytamoxifen (4-OHT), fulvestrant, raloxifen and CMP8, on GFP-ERLID fusion protein stability in NIH3T3 cells, where the GFP-ERLBD fusion protein was the wild type (SEQ ID NO: 3; open bars) or a 4 point mutation of SEQ ID NO: 3 (cross-hatched bars) expressed in NIH3T3 cells in the absence of any ligand (control) and in the presence of 4-hydroxytamoxifen (4-OHT), rolaxifen, fluvestrant and CMP8.

FIGS. 7B-7C are graphs that show the stability of GFP-ERLBD (SEQ ID NO: 3), as indicated by median fluorescence intensity (MFI), as a function of amount, in nM, of 4-hydroxytamoxifen (4-OHT) (FIG. 7B) and estradiol (FIG. 7C)

### BRIEF DESCRIPTION OF THE SEQUENCES

SEQ ID NO: 1 is an amino acid sequence of a degron-containing peptide,

TRGVEEVAAIPEVLRRRGN.

SEQ ID NO: 2 is a degron tetrapeptide, RRRG.

SEQ ID NO: 3 is a sequence of referred to herein as the Estrogen Receptor Ligand Binding Domain (ERLBD)-based LID system (also called “ER-LID”):

SLALSLTADQMVSALLDAEPPILYSEYDPTRPFSEASMMGLLTNLADRE
LVHMINWAKRVPGFVDLTLHDQVHLLECAWLEILMIGLVWRSMEHPGKL
LFAPNLLLDRNQGKCVEGMVEIFDMLLATSSRFRMMNLQGEEFVCLKSI
ILLNSGVYTFLSSTLKSLEEKDHIHRVLDKITDTLIHLMAKAGLTLQQQ
HQRLAQLLLILSHIRHMSNKGMEHLYSMKCKNVVPLYDLLLEMLDAHRL
GGGSGGGSTRKHKILHRLLQDSSRRRGN.

SEQ ID NO: 4 is a peptide KHKILHRLLQDSS.

SEQ ID NO: 5 is a degron peptide, RRRGN.

SEQ ID NO: 6 is a degron peptide, YALAA.

SEQ ID NO: 7 is the estrogen receptor ligand binding domain, corresponding to residues 305-549 of human estrogen receptor 1, AAI28574:

SLALSLTADQMVSALLDAEPPILYSEYDPTRPFSEASMMGLLTNLADRE
LVHMINWAKRVPGFVDLTLHDQVHLLECAWLEILMIGLVWRSMEHPGKL
LFAPNLLLDRNQGKCVEGMVEIFDMLLATSSRFRMMNLQGEEFVCLKSI
ILLNSGVYTFLSSTLKSLEEKDHIHRVLDKITDTLIHLMAKAGLTLQQQ
HQRLAQLLLILSHIRHMSNKGMEHLYSMKCKNVVPLYDLLLEMLDAHRL

SEQ ID NO: 8 is the synthetic sequence GGGSGGGS.

SEQ ID NO: 9 is a peptide KHKILHRLLQD.

SEQ ID NO: 10 is the human influenza hemagglutinin (HA) amino acid sequence YPYDVPDYA.

### DETAILED DESCRIPTION

Described herein are compositions, systems and methods for modulating the stability and function of proteins rapidly and reversibly, in vitro and in vivo, through the administration of cell-permeable small molecules to cultured cells or living animals. The coding sequence for a protein of interest (POI) is genetically fused to a sequence encoding a stability-affecting protein domain capable of interacting with a small-molecule ligand, the presence, absence, or amount of which ligand modulates the stability of the fusion protein. These compositions, systems and methods are designed to provide (1) protein domains in the form of cDNA constructs that, when fused to any gene-of-interest, can be used by investigators to degrade proteins-of-interest; and (2) cell-permeable small molecules that bind to and stabilize or destabilize the DD or LID domains thereby restoring or eliminating the function of the protein-of-interest.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. Definitions

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

When a range of values is provided, it is understood that each intervening value, to the tenth of the unit of the lower limit unless the context clearly dictates otherwise, between the upper and lower limits of that range is also specifically disclosed. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm. Each smaller range between any stated or intervening value in a stated range and any other stated or intervening value in that stated range is encompassed by the disclosure. The upper and lower limits of the smaller ranges may be independently included or excluded in the range, and each range where either, neither or both limits are included in the smaller ranges is also encompassed by the disclosure, subject to any specifically excluded limit in the stated range. Where the stated range includes one or both of the limits, ranges excluding either or both of those included limits are also included.

Examples of other compositions, systems and methods for conditional regulation of protein levels and function are described in U.S. Pat. No. 8,173,792 and U.S. Patent Pre-Grant Publications 2010/0034777 and 2012/0178168, each of which is herein incorporated by reference in its entirety.

As used herein, a “ligand-induced destabilizing” (LID) domain (a.k.a., a “ligand-induced degradation” (LID) domain), a “stability-affecting domain” or “stability-affecting protein domain capable of interacting with a small-molecule ligand” may be a single polypeptide that functions as a stability-affecting protein domain capable of interacting with a small-molecule ligand, the presence, absence, or amount of which is used to modulate the stability of the fusion protein, as described herein. Such a destabilization domain may or may not require the interaction of another protein for modulating stability of the POI. In some embodiments, the ligand-regulated, stability-affecting domain merely requires exposure to a small molecule ligand, and does not require formation of a ternary complex, as does the FKBP-rapamycin-FRB complex. An exemplary species is a “single-domain,” ligand-dependent destabilization domain, wherein the single polypeptide comprises only a single domain (i.e. folded structure or functional unit as determined by X-ray crystallography, protease digestion, computer modeling, etc.).

The stability-affecting polypeptide domain can be used in the system disclosed herein to stabilize or destabilize a protein of interest (POI) depending on which ligand is used in the system. In some embodiments, the stability-affecting polypeptide domain acts as a degradation domain (DD)/ligand-induced stability domain, for example in the presence of estradiol as a ligand. In some embodiments, the stability-affecting polypeptide domain acts as a ligand-induced destabilization domain, for example in the presence of tamoxifen as a ligand.

As used herein, the terms “protein” and “polypeptide” are used interchangeably and without distinction to refer to a compound made up of a chain of amino acid residues linked by peptide bonds. Unless otherwise indicated, the sequence for peptides is given in the order from the “N” (or amino) terminus to the “C” (or carboxyl) terminus. It is understood that polypeptides include a contiguous sequence of amino acid residues.

As used herein, a “protein of interest” or “POI” is any protein, or functional fragment or derivative thereof, that one skilled in the art wishes to study, or for which one desires to conditionally destabilize and regulate the degradation of the protein, functional fragment or derivative thereof.

As used herein, a “conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art.

Two amino acid sequences or two nucleotide sequences are considered “homologous” if they have an alignment score of >5 (in standard deviation units) using the program ALIGN with the mutation gap matrix and a gap penalty of 6 or greater (Dayhoff, M. O., in Atlas of Protein Sequence and Structure (1972) Vol. 5, National Biomedical Research Foundation, pp. 101-110, and Supplement 2 to this volume, pp. 1-10). The two sequences (or a sequence of a specified length of contiguous amino acid residues from each of the two sequences) are homologous if their amino acids are greater than or equal to 50%, 70%, 80%, 85%, 90%, 91%, 92%, 93%, 94%, 95%, 96%, 97%, 98% or 99% identical when optimally aligned using the ALIGN program mentioned above.

A peptide or peptide fragment is “derived from” a parent peptide or polypeptide if it has an amino acid sequence that is homologous to, but not identical to, the parent peptide or polypeptide, or of a conserved fragment from the parent peptide or polypeptide.

As used herein, a “variant” protein is a protein having an amino acid sequence that does not occur in nature, as exemplified by sequences in GenBank.

As used herein, a “mutant” is a mutated protein designed or engineered to alter properties or functions relating to protein stabilization and/or the ability to act as a stability-affecting protein domain capable of interacting with a small-molecule ligand, the presence, absence, or amount of which is used to modulate the stability of the fusion protein.

As used herein, the terms “domain” and “region” are used interchangeably herein and refer to a contiguous sequence of amino acids within a POI or destabilizing domain, typically characterized by being either conserved or variable and having a defined function, such as being affected its ability to interact with a small-molecule ligand, the presence, absence, or amount of which is used to modulate the stability of the fusion protein, conferring stability or instability, enzymatic function, etc.

As used herein, a “degron” is an amino acid sequence that interacts with the cellular protein degradation machinery and specifies degradation of itself and any fusion protein of which it is a part. In the disclosed methods for designing proteins that display ligand-dependent instability as well as specific compositions of matter, certain other degrons may be useful in place of the mammalian degron (RRRG; SEQ ID NO: 2), such as the bacterial YALAA peptide (SEQ ID NO: 6) and/or the yeast CL1 degron. Both RRRG (SEQ ID NO: 2) and RRRGN (SEQ ID NO: 5) have been tested and found to be efficient degrons, although RRRG is somewhat stronger. Thus, a peptide RRRG (SEQ ID NO: 2), optionally having a fifth residue is contemplated.

As used herein, “preferentially binds” means to bind with greater efficiency to a subject molecule (such as a particular amino acid sequence) than another molecule. The difference in binding efficiency may be 5-fold, 10-fold, 50-fold, 100-fold, 500-fold, 1.000-fold, 10,000 fold, or more.

As used herein, “introduction of nucleic to cells” means transfection, transduction (infection), or transformation of nucleic acids (e.g., DNA) into cells, such that the nucleic acids may be used by the cell to express a protein of interest.

As used herein, “degradation” or “destruction” of a protein means its hydrolysis into smaller proteins or amino acids, such as by the cellular proteosome.

As used herein, “fused” means arranged in-frame as part of the same contiguous sequence of amino acids in a polypeptide. Fusion can be direct such there are no additional amino acid residues or via a linker to improve performance or add functionality.

As used herein, “modulate” intends a decrease, an increase, or some other measurable change, e.g., in the levels, stability or biological function of a protein.

### II. Exemplary Systems and Compositions

The present composition, system and method relate to the conditional stabilization of a protein of interest (POI) using a system comprising a nucleic acid sequence encoding a fusion protein comprising a POI fused in-frame to a ligand-regulated, stability-affecting domain, and a ligand. Upon introduction of the nucleic acid sequence to a cell, the fusion protein is expressed and the stability of the fusion protein can be degraded upon administration of the ligand. The stability-affecting polypeptide, also referred to as the ligand-regulated domain or “ligand-induced degradation” (LID) domain, can be preselected to confer either stability or instability to the entire fusion protein, depending on the presence or absence of a particular ligand that is not otherwise toxic to host cells or host organism.

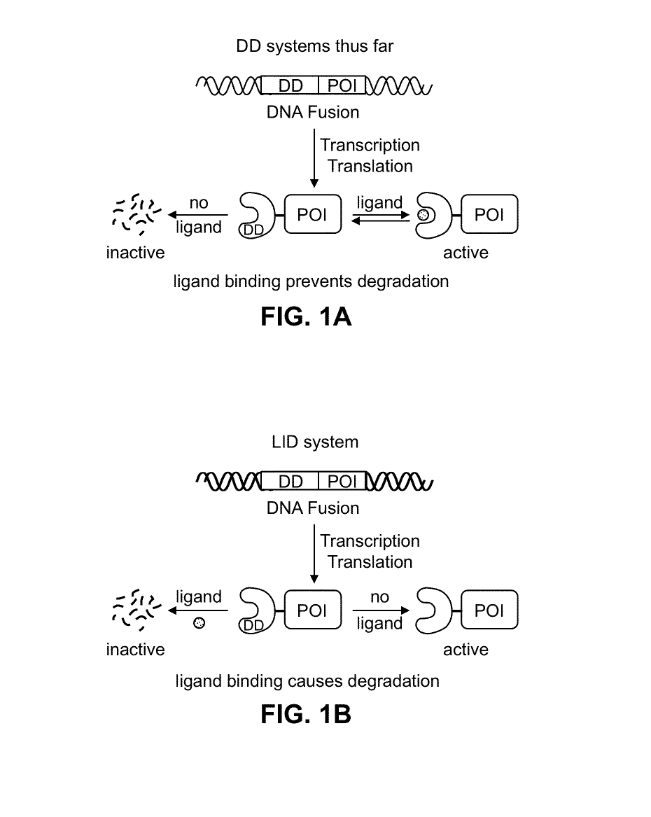

The technology described herein fundamentally reverses previously described technologies where a small molecule ligand, such as Shield-1, stabilizes a fusion protein of a destabilizing domain and a protein of interest. FIG. 1A shows a prior art technology, where a destabilizing domain (DD) fused to a protein-of-interest (POI) in which the POI is degraded in the absence of ligand; addition of ligand to the system prevents degradation of the target protein.

In the presently described systems, methods and compositions a fusion protein of a destabilizing domain and a protein of interest small molecule ligand is stable in the absence of a ligand and becomes unstable in the presence of the ligand, as illustrated in FIG. 1B. FIG. 1B shows an engineered “ligand-induced degradation” (LID) domain that is genetically fused to a POI, where expression of the fusion construct results in a stable fusion protein in the absence of ligand; the addition of a high-affinity ligand causes rapid and processive degradation of the LID domain and fusion protein. Accordingly, the present system allows for the rapid and reversible destabilizing a target protein in vitro or in vivo allow an investigator to make, for example, a knock-in mouse where a specific gene-of-interest is fused to a “ligand-induced destabilizing” (LID) domain. With this technology, in the absence of the ligand and assuming that fusion of the LID domain to the protein does not interfere with its normal function, the mouse develops normally. At a suitable time, the investigator administers the ligand, thereby causing degradation of the test protein. The method is more rapid than methods that regulate DNA or mRNA expression, as the protein is directly targeted for degradation.

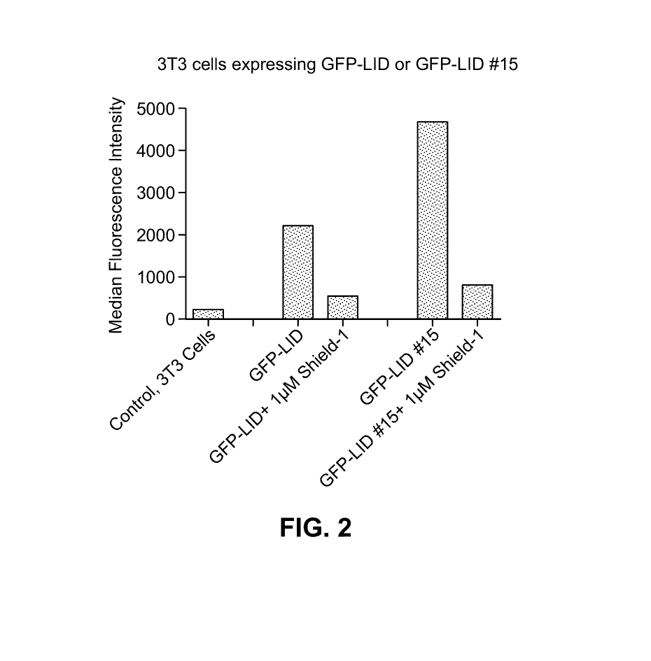

In studies conducted in support of the invention, and with reference to the Materials and Methods set forth in the Examples section below, the stabilities of two LID domain fusion proteins, a first generation construct based on the FKBP protein, here called “GFP-LID” and a next-generation construct having improved characteristics, here called “GFP-LID #15” were compared. FIG. 2 shows the stabilities of these fusion proteins in the presence and absence of the small molecule ligand, Shield-1. For this study, NIH3T3 fibroblast cells were infected with retrovirus expressing GFP-LID or GFP-LID #15. Transduced NIH3T3 cells were treated with or without 1 μM Shield-1 for 24 hours. The fluorescence intensities of these GFP fusions were analyzed by flow cytometry. The median fluorescence intensity (MFI) was used to determine the stability of the GFP fusion proteins. In the absence of Shield-1, the GFP-LID #15 fusion protein is more stable than the first generation (GFP-LID), and upon addition of Shield-1, both LID#15 and LID fusion proteins were destabilized.

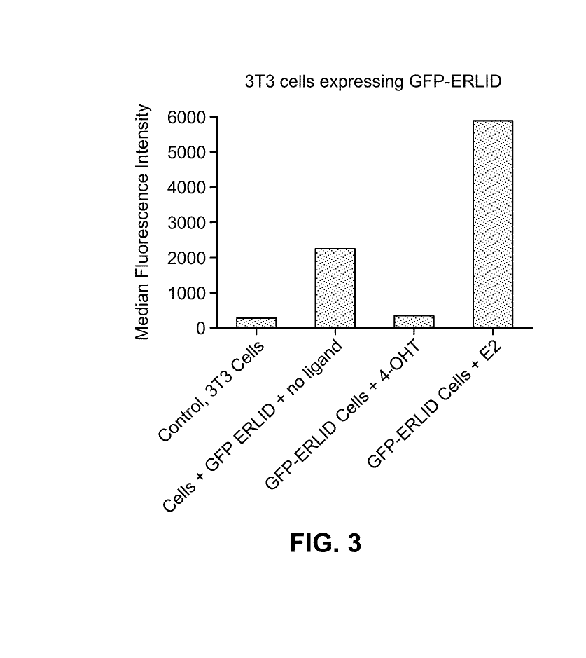

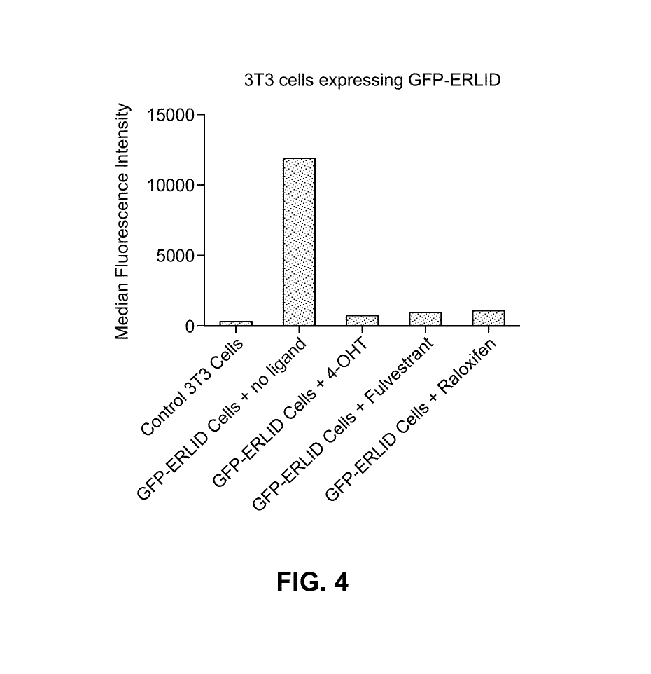

In other studies, an alternative ligand-responsive, stability-affecting polypeptide domain system based on the estrogen receptor ligand-binding domain (ERLBD) was investigated. In this system, in the absence of any ligand the ERLID fusion protein is stable. When an estrogen antagonist, such as tamoxifen, is added, a significant decrease in stability (i.e. an increase in instability) is observed. Conversely, upon addition of an estrogen agonist, enhanced/increased stability (i.e., a decrease in instability) is observed. Thus, a single genetic construct (the ERLID) fused to any POI can be used to downregulate or upregulate the expression levels of a POI, depending on whether and antagonist or agonist is used, respectively. An antagonist can be used to knock-down protein levels, and an agonist can be used to raise protein levels. This is illustrated in the studies conducted and data shown in FIG. 3 and FIG. 4.

Specifically, FIG. 3 demonstrates the stability of the GFP-tagged ERLID fusion protein in the presence of various small molecule ligands. NIH3T3 cells were infected with retrovirus expressing GFP-ERLID. NIH3T3 cells stably expressing GFP-ERLID were then treated with 3 μM 4-hydroxytamoxifen (4-OHT), 2 μM estradiol (E2) or with only vehicle for 24 hours. The fluorescence intensities of the GFP fusion proteins were analyzed by flow cytometry, and MFI was used to determine the stability of GFP fusion proteins. Without addition of ligand, the GFP-ERLID is stable. Addition of an estrogen receptor antagonist, such as 4-OHT, induces degradation of GFP-ERLID fusion protein. Conversely, the addition of an estrogen receptor agonist, such as estradiol (E2), stabilizes the GFP-ERLID fusion protein.

FIG. 4 shows the effects of various estrogen receptor antagonists on GFP-ERLID fusion protein stability. Treating NIH3T3 cells stably expressing the GFP-ERLID fusion protein with 10 μM fulvestrant or 5 μM raloxifene induced degradation of GFP-ERLID fusion protein.

Posttranslational regulation of protein abundance in cells is a powerful tool to study protein function. The single protein regulatory domain identified herein and referred to as ERLID is degraded upon exposure to a small-molecule ligand. Genetic fusion of this domain to a protein of interest allows the rapid, ligand-induced modulation of the fusion protein in cells and higher organisms.

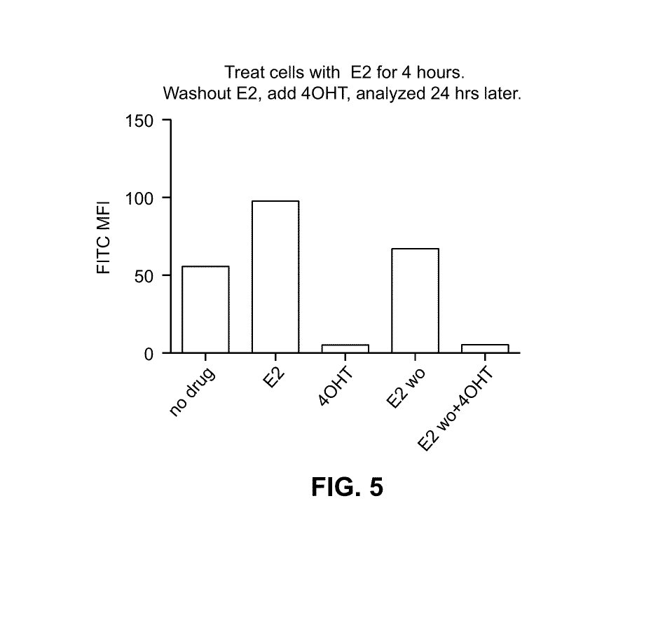

As mentioned above, and system described herein allows the level of the fusion protein to be reversibly controlled on a posttranslational level by a ligand with high specificity for the LID domain. The reversibility of the system is demonstrated in the data shown in FIG. 5. Using median fluorescence intensity as an indicator of the presence of stable fusion protein GFP-ERLIP, NIH3T3 cells expressing the fusion protein in the absence of a ligand was measured (no drug). The level of expression of the fusion protein after exposure for 4 hours to estradiol (E2) and after exposure to 4-hydroxytamoxifen (4-OHT) were measured. Estradiol stabilizes the fusion protein, as evidenced by the increased MFI, while 4-OHT acts as an antagonist that destabilizes the fusion protein, as evidenced by the decrease in MFI. Removal of estradiol by washing the cells (E2 wo) results in a reduced MFI relative to that observed in the presence of E2, indicating destabilization. Addition of 4-hydroxytamoxifen to the wash solution (E2 wo+4OHT) destabilizes the fusion protein. This study demonstrates the ability to control the level or extent of stability or destabilization of the fusion protein by controlling the amount and type of ligand introduced to the system.

The amino acid sequence of the “ligand-induced degradation” (LID) domain is TRGVEEVAAIPEVLRRRGN (SEQ ID NO: 1). This LID domain has a degron sequence which can bind intramoleculary to a FKBP12 protein and causes the degron to be hidden from the cellular protein degradation machinery. When the LID domain is fused to the C-terminus of a target protein, a ligand such as Shield-1 can be used to bind to the same binding site as the 19-amino acid peptide. Addition of Shield-1 thereby replaces and exposes the degron causing degradation of the LID domain and the fusion protein. Accordingly, in one embodiment, the degron-containing peptide identified herein as SEQ ID NO: 1 is fused to the C-terminus of the FKBP12 protein in a “ligand-induced degradation” (LID) system.

A small four-amino acid peptide degradation tag (a.k.a “degron”), having the amino acid sequence RRRG (SEQ ID NO: 2) has been reported (Bonger, K. M., Chen, L.-c., Liu, C. W. & Wandless, T. J. Nat. Chem. Biol. 7, 531-537 (2011)) and shown to be useful as a C-terminal fusion to a protein of interest (POI) which results in rapid proteasome-mediated degradation in mammalian cells. This peptide degron can be fused in-frame to the C-terminal alpha helix of a POI such as the yellow fluorescent protein (YFP), causing the expressed YFP to be strongly destabilized. Another degron, RRRGN (SEQ ID NO: 5), can also be used in the disclosed system and methods. Both RRRG (SEQ ID NO: 2) and RRRGN (SEQ ID NO: 5) have been tested and found to be efficient degrons, and thus, the RRRG (SEQ ID NO: 2), degron may optionally have a fifth residue. By tagging any protein with the RRRG degron (SEQ ID NO: 2), or with the slightly weaker degron RRRGN (SEQ ID NO: 5), at a protein's C-terminus allows for a degradative response.

In one embodiment, a ERLBD-based-LID system is comprised of a nucleic acid that encodes for, or a protein having the following elements operatively connected from N- to C-terminus: (1) an estrogen receptor ligand-binding domain (residues 305-549 of the human estrogen receptor 1, AAI28574), followed by (2) an optional spacer peptide, typically having between 2-20, 4-20, 4-18, 4-16, 4-14, 4-12, 4-10, 4-8, 6-8 or 8 amino acid residues, followed by (3) a peptide having at least about 60%, 70%, 80%, 85%, 90%, or 95% sequence identity to KHKILHRLLQDSS (SEQ ID NO: 4), optionally followed by 2 amino acids created from a restriction site (e.g., SS), then (4) a degron, such as RRRGN (SEQ ID NO: 5) or RRRG (SEQ ID NO: 2), or any degron having the sequence RRRG (SEQ ID NO: 2) and an optional fifth residue at the N- or C-terminus. The system will include, in addition to a nucleic acid encoding for the protein with elements (1)-(4) or the protein with elements (1)(4), a cell permeable ligand. An exemplary ERLBD-based LID protein sequence is identified herein as SEQ ID NO: 3.

Truncation and mutation studies of the 19-amino acid peptide identified herein as SEQ ID NO: 1 revealed that the shortest possible degron is the tetrapeptide RRRG (SEQ ID NO: 2). When this tetrapeptide is fused at the C-terminus of the otherwise stable yellow fluorescent protein (YFP), it results in a strongly enhanced degradation and diminution/abolition of fluorescence in 3T3 cells. Thus, a ligand-inducible degradation system was developed by fusing RRRG (SEQ ID NO: 2) to a POI (see FIG. 1B). This confirms the potential of the LID domain to reversibly regulate levels and/or function of a protein of interest in cells or organisms.

This technology benefits researchers on several fronts. First, it is general and can be applied to any protein of interest. Second, only one genetic fusion is needed in contrast to other previously described technologies that need multiple protein domains. Lastly, the method and system described herein allow protein levels to be rapidly and reversibly controlled on a posttranslational level by a ligand with high specificity for the LID domain. In addition, the fact that this technology uses only one domain to regulate protein levels may facilitate its use in transgenic animals. The system and method employing stability-affecting protein domain described herein are easily adaptable for use in other eukaryotic cells, tissues and model organisms, e.g., cultured eukaryotic cells (including mammalian and specifically human cells); yeasts (including, for example, S. cerevisiae, C. albicans and S. pombe); mammalian embryos and fertilized or unfertilized oocytes; rodents; nematodes; zebrafish; flies such as Drosophila species; and frogs such as Xenopus species).

These new compositions, systems and methods differ from those previously described, as an investigator can make a knock-in mouse where a specific gene-of-interest is fused to a nucleic acid sequence encoding the LID domain described herein, for rapid and reversible destabilization of a protein-of-interest (POI), in vitro or in vivo. For example, in the absence of a ligand such as Shield-1, the mouse expressing the fusion protein (in which the LID domain does not interfere with the protein's normal function) develops normally, but upon administration of the ligand at an appropriate time, the POI is rapidly degraded. This degradation is easily reversed by removal of the ligand from the system. For example, the ligand may be administered regularly from an early age (including in utero) to stabilize the fusion protein until the mice achieve a specified age, at which time withdrawal of the ligand results in a the rapid degradation of the fusion protein. Unlike Cre-mediated gene disruption, the presently described method is reversible, simply by reinitiating the administration of the ligand, allowing the rapid, reversible, and conditional control of protein function in a complex system.

It is possible to engineer this type of ligand-induced instability into any protein-ligand combination. The present disclosure describes several LID systems which have been tested with various genes and target proteins, and the LID system is robust and generally applicable to mammalian cells and organisms. Although it was possible that the degron (such as the RRRG; (SEQ ID NO: 2)), optionally with a fifth residue, such as RRRGN (SEQ ID NO: 5)) used in such systems might have only been recognized for degradation in mammalian or metazoan cells, the presently described LID system has been applied to several proteins experimentally, and like DD technology, this system has proven to be useful in a wide variety of organisms. Furthermore, this LID system provides a much-needed improvement having a larger dynamic range (e.g., the difference in protein levels between the ligand and no-ligand states). In contrast to other systems, the presently described LID system does not show instability in the presence of malaria parasites.

Ideal techniques for conditionally stabilizing biological macromolecules are specific, fast, reversible, and tunable. Cell-permeable small molecules often deliver the latter three features but, apart from a few well-known exceptions, cell-permeable small molecules are typically not specific for a single biological target. The ideal conditional stabilization technology combines the specificity of reverse genetics (i.e., well-defined DNA changes in a large genomic background) with the conditionality of cell-permeable small molecules.

The present systems work in different cell types, and works in cell culture as well as in animals. The system provides heretofore unprecedented control of the levels of preselected protein in cells, with excellent dose and temporal control. While the present methods have been described with reference to a “ligand-induced degradation” (LID) domain having SEQ ID NO: 3, other conditional stabilizing/destabilizing domains and other ligands may be used, and other examples are set forth hereinbelow. Preferred stability-affecting proteins modulate the degradation of a fusion protein in the presence of ligand, as determined, for example, using the kinetic and immunological assays described herein.

The abundance of variants obtained in the screens, as well as the ability to use different ligand-regulated stability affecting polypeptide domains, suggests that further refinements in screening may lead to additional stability-affecting proteins selected for various properties (e.g., rate of degradation, potency of stabilization, subcellular localization, and the like). Moreover, the stability-affecting proteins work when fused to either the N- or the C-terminus of a POI, illustrating the modularity of the components of the system.

Preferred stability-affecting proteins produce a 5, 10, 20, 30, 40, 50, 60, or more-fold difference in the levels of a preselected POI, which difference can be detected in cell or in an animal in the presence, absence, or amount of a small-molecule ligand, which is used to modulate the stability of the fusion protein. In some embodiments, the gene or allele encoding the naturally-occurring POI (i.e. the native protein, not a fusion protein) is deleted or disrupted in the genome of the cells or animal in which the conditional protein stability system is used or replaced by a DNA encoding the fusion protein. In this manner, the only source of the POI is the conditionally stabilized fusion protein, allowing its function to be studies in the absence of the interfering wild-type/naturally-occurring protein.

The stability-affecting proteins may encompass amino acid substitutions that do not substantially affect stability, including conservative and non-conservative substitutions Preferably, the amino acid sequences of the peptide inhibitors encompassed in the invention have at least about 60% identity, further at least about 70% identity, preferably at least about 75% or 80% identity, more preferably at least about 85% or 90% identity, and further preferably at least about 95% identity, to the amino acid sequences set forth herein. Percent identity may be determined, for example, by comparing sequence information using the advanced BLAST computer program, including version 2.2.9, available from the National Institutes of Health. The BLAST program is based on the alignment method of Karlin and Altschul ((1990) Proc. Natl. Acad. Sci. U.S.A. 87:2264-68) and as discussed in Altschul et al. ((1990) J. Mol. Biol. 215:403-10; Karlin and Altschul (1993) Proc. Natl. Acad. Sci. U.S.A. 90:5873-77; and Altschul et al. (1997) Nucleic Acids Res. 25:3389-3402).

Conservative amino acid substitutions may be made in the amino acid sequences described herein to obtain derivatives of the peptides that may advantageously be utilized in the present invention. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, amino acids having acidic side chains, such as aspartic acid and glutamic acid, can often be substituted with amino acids having amide side chains, such as asparagine and glutamine.

The stability-affecting proteins may be fragments of the above-described destabilizing domains, including fragments containing variant amino acid sequences. Such fragments are readily identified using the assays described herein. In some embodiments, the fragments retain the ability to undergo a structural change upon exposure to the ligand, with similar efficiency to the destabilizing domains described herein or with at least 90% efficiency, at least 80% efficiency, at least 70% efficiency, or even at least 50% efficiency with respect to the described stability-affecting proteins.

Methods to rapidly and reversibly perturb the functions of specific proteins are desirable tools for studies of complex biological processes. An experimental strategy, as described herein, was designed to regulate the intracellular concentration of any protein of interest by using an engineered destabilizing protein domain and a cell-permeable small molecule. Destabilizing domains have general utility to confer instability to a wide range of proteins including integral transmembrane proteins. The present disclosure presents a ligand-induced destabilizing domain system based on the ligand binding domain of the estrogen receptor, which can be regulated by one of two synthetic ligands, CMP8 (Kinzel, O. et al., J. Med. Chem., (2006), 49:5404-5407) or 4-hydroxytamoxifen. (Miyazaki, et al., (2012) J. Am. Chem. Soc.; 134 (9):3942-5).

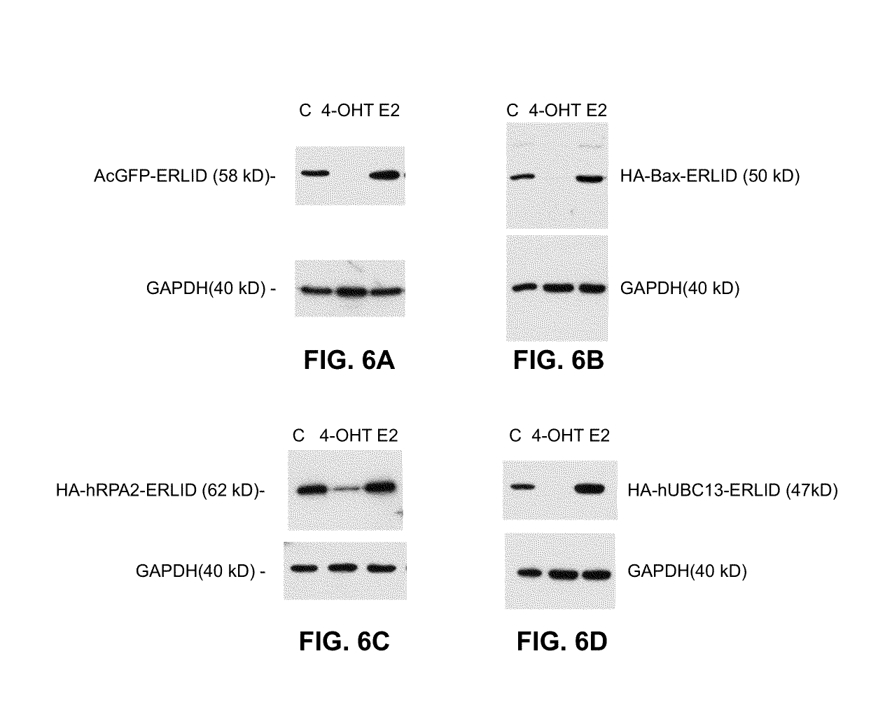

To illustrate that the destabilizing domain ERLID can be used in conjunction with any protein of interest, the domain was used to regulate the stability of three other proteins, BAX (“apoptosis regulator BAX [Mus musculus]” (GenBank nucleotide: NM_{—}007527, Genbank Protein: NP_{—}031553.1), RPA2 (“replication protein A 32 kDa subunit isoform 1 [Homo sapiens]” GenBank nucleotide: NM_{—}002946.4, Genbank Protein: NP_{—}002937.1) and UBC13 (“Ubiquitin-conjugating enzyme E2N (UBC13 homolog, yeast) [Homosapiens]”, GenBank nucleotide: BC108704, GenBank Protein: AAI08705.1). Following the same methodologies described in the Materials and Method section below, the fusion proteins were expressed in NIH3T3 cells, and Western blots were used to inspect the level of fusion protein expressed. The results are shown in FIGS. 6A-6D. The expression of stable fusion protein GFP-ERLID is shown in FIG. 6A, of HA-Bax-ERLID in FIG. 6B, of HA-hRPA2-ERLID in FIG. 6C and of HA-hUBC13-ERLID in FIG. 6D (lanes in each figure labelled “C”). The notation HA corresponds to human influenza hemagglutinin (HA) amino acid sequence YPYDVPDYA (SEQ ID NO: 10). In the presence of 4-hydroxytamoxifen (4-OHT) the fusion proteins are destabilized (lane in each figure labelled “4-OHT”). In the presence of estradiol (lane in each figure labelled “E2”), each of the fusion proteins is stabilized, and based on a comparison of the E2 lane with the control lane, and in view of the leveling control, GAPDH, it is apparent that E2 serves to increase the level of stability of the fusion proteins.

Several estrogen receptor (ER) antagonists were investigated. In some embodiments, the LID domain comprises SEQ ID NO: 3 or a sequence having at least about 80%, 85%, 90%, or 95% sequence identity to SEQ ID NO: 3, and having the following elements (listed from N- to C-terminus): (1) an ER ligand-binding domain (residues 305-549 of the human estrogen receptor 1, GenBank Accession AAI28574), or an ER ligand-binding domain having at least about 80%, 85%, 90%, 95% or 98% sequence identity to residues 305-549 of the human estrogen receptor 1, GenBank Accession AAI28574), followed by (2) an optional peptide spacer having between 2-20, 4-20, 4-10, 6-10, or 8 amino acid residues, followed by (3) a peptide having at least about 80%, 85%, 90% or 95% sequence identity to KHKILHRLLQDSS (SEQ ID NO: 4) or to KHKILHRLLQD (SEQ ID NO: 9), followed by (4) a degron peptide, such as SEQ ID NO: 2, SEQ ID NO: 5, SEQ ID NO: 6, or a peptide having at least about 80%, 85%, 90% or 95% sequence identity to SEQ ID NO: 2, SEQ ID NO: 5, or SEQ ID NO: 6.

Both RRRG (SEQ ID NO: 2) and RRRGN (SEQ ID NO: 5) have been tested and found to be efficient degrons, although RRRG is somewhat stronger. Thus, a peptide RRRG (SEQ ID NO: 2), optionally having a fifth residue is contemplated.

In some embodiments, commercially available 4-hydroxytamoxifen serves as a ligand for the destabilizing domain. Additionally, a mutated version of the ERLBD (element 1 above), in which 3 or 4 mutations that allow the use of a synthetic ligand known as “CMP8” (Kinzel, O. et al., J. Med. Chem., (2006), 49:5404-5407; (9a-(4-Chlorobenzyl)-7-hydroxy-4-[4-(2-piperidin-1-ylethoxy)phenyl]-1,2,9,9a-tetrahydro-3H-fluoren-3-one)) were introduced and investigated as part of the presently described system. Because the CMP8 ligand does not bind well to wild-type ER LBDs, it is more “biologically silent,” (and thus innocuous) to use as a ligand. The mutants that displayed drug-dependent stability were designed using yellow fluorescent protein (YFP) or green fluorescent protein (GFP) as a reporter for estrogen receptor ligand binding domain (residues 305-549 of estrogen receptor-1, ERS1). Clones with four mutations and with six mutations, T371A, L384M, M421G, N519S, G521R and Y437S, yielded a large dynamic, relative to the parent ESR1 sequence, of stability in the presence of CMP8.

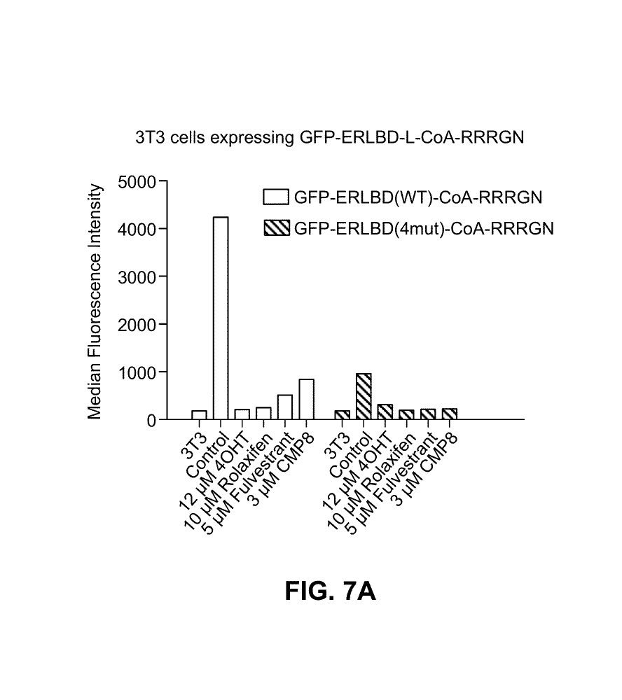

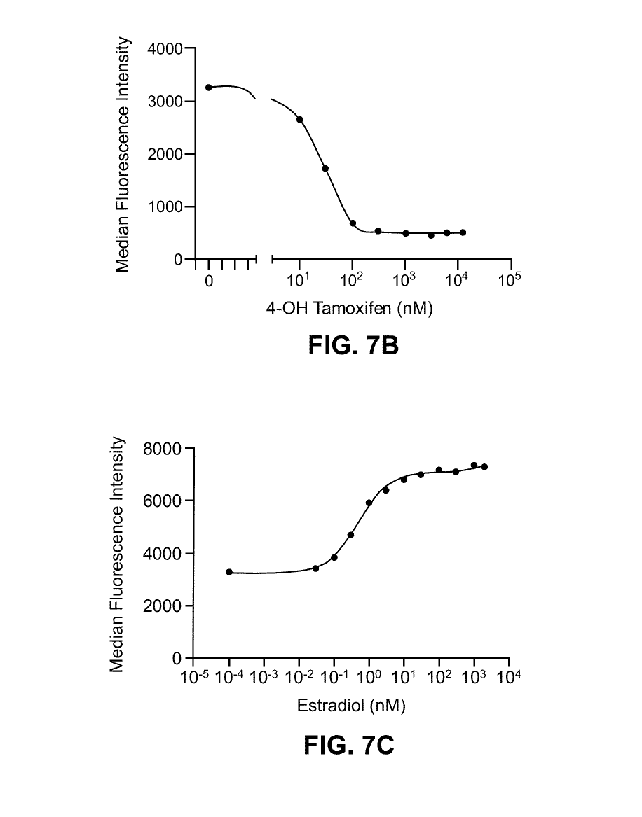

FIG. 7A shows the results of a study where the stability of fusion proteins was modulated using several antagonist ligands. The fusion proteins were expressed in NIH3T3 cells, where in one study the expressed fusion protein was that identified by SEQ ID NO: 3 fused in frame to GFP, and in another study the expression fusion protein was comprised of a four point mutant of the estrogen receptor ligand binding domain (residues 305-549 of estrogen receptor-1) where mutated residues were: L384M, M421G, G521R and Y437S fused to GFP. The degron sequence was RRRGN (SEQ ID NO: 5). Stability of GFP-ERLID fusion protein in NIH3T3 cells was ascertained using median fluorescence intensity, where the stability of the GFP-ERLID fusion protein in the cells in the absence of any ligand is shown (open bar, control) and in the presence of the indicated amount of 4-hydroxytamoxifen (4-OHT), rolaxifen, fluvestrant and CMP8.

With continuing reference to FIG. 7A the stability of the mutant fusion protein (cross-hatched bars) in the absence of any ligand (cross-hatched bar, control) and in the presence of 4-hydroxytamoxifen (4-OHT), rolaxifen, fluvestrant and CMP8 was measured. The ligands antagonize both fusion proteins, causing destabilization of the fusion protein.

FIGS. 7B-7C are graphs that show the dose dependent destabilization of GFP-ERLBD (SEQ ID NO: 3) as a function of amount of ligand. FIG. 7B shows the destabilization of the fusion protein with an increasing amount of 4-hydroxytamoxifen (4-OHT) and FIG. 7C shows the increasing stabilization of the fusion protein with an increasing amount of estradiol.

Accordingly, in another embodiment, a system comprising a LID domain comprised of the following elements (listed from N- to C-terminus) is contemplated: (1) a mutant ER ligand-binding domain, consisting of residues 305-549 of the human estrogen receptor 1 (GenBank Accession AAI28574) with three or more mutations selected from T371A, L384M, M421G, N519S, G521R and Y437S, followed by (2) an optional peptide spacer having between 2-20, 4-20, 4-10, 6-10, or 8 amino acid residues, followed by (3) a peptide having at least about 80%, 85%, 90% or 95% sequence identity to KHKILHRLLQDSS (SEQ ID NO: 4) or to KHKILHRLLQD (SEQ ID NO: 9), followed by (4) a degron peptide, such as SEQ ID NO: 2, SEQ ID NO: 5, SEQ ID NO: 6, or a peptide having at least about 80%, 85%, 90% or 95% sequence identity to SEQ ID NO: 2, SEQ ID NO: 5, or SEQ ID NO: 6. The system comprises CMP8 or another synthetic ligand with activity to destabilize the LID domain when fused to a protein-of-interest. In one embodiment, the optional peptide spacer is SEQ ID NO: 8 or a peptide having at least about 80%, 85%, 90% or 95% sequence identity to SEQ ID NO: 8.

In still another embodiment, a system comprising a LID domain comprised of the following elements (listed from N- to C-terminus) is contemplated: (1) a mutant ER ligand-binding domain having at least about 95% sequence identity to residues 305-549 of the human estrogen receptor 1 (GenBank Accession AAI28574), followed by (2) an optional peptide spacer having between 2-20, 4-20, 4-10, 6-10, or 8 amino acid residues, followed by (3) a peptide having at least about 80%, 85%, 90% or 95% sequence identity to KHKILHRLLQDSS (SEQ ID NO: 4) or to KHKILHRLLQD (SEQ ID NO: 9), followed by (4) a degron peptide, such as SEQ ID NO: 2, SEQ ID NO: 5, SEQ ID NO: 6, or a peptide having at least about 80%, 85%, 90% or 95% sequence identity to SEQ ID NO: 2, SEQ ID NO: 5, or SEQ ID NO: 6. The system comprises CMP8 or another synthetic ligand with activity to destabilize the LID domain when fused to a protein-of-interest. In one embodiment, the optional peptide spacer is SEQ ID NO: 8 or a peptide having at least about 80%, 85%, 90% or 95% sequence identity to SEQ ID NO: 8.

### III. Methods, Cells and Transduced Animals

Also disclosed is a method for conditionally stabilizing a protein of interest, comprising fusing a nucleic acid encoding the protein of interest in-frame to a nucleic acid encoding a “ligand-induced destabilization/degradation” (LID) domain capable of interacting with a small-molecule ligand, to produce a nucleic acid encoding a fusion protein; introducing the nucleic acid encoding the fusion protein into a cell; expressing the fusion protein in the cell; and exposing the cell to a small-molecule ligand, the presence, absence, or amount of which is used to modulate the stability of the fusion protein. In some embodiments, an improved FKBP12-based “ligand-induced destabilizing” (LID) domain is provided. In some embodiments, the LID domain comprises SEQ ID NO: 1. In some embodiments, the LID domain is SEQ ID NO: 1. In some embodiments, an ERLBD-based LID system is provided. In some embodiments, the system comprises a ligand-binding domain; an optional spacer; an optional 13-residue peptide (KHKILHRLLQDSS; herein identified as SEQ ID NO: 4) that includes 2 amino acids created from a restriction site; and a degron. A ligand that destabilizes the fusion protein is included in the system. In some embodiments, a fusion protein is provided, wherein the fusion protein comprises SEQ ID NO: 1. In some embodiments, a fusion protein is provided, wherein the fusion protein comprises SEQ ID NO: 3. In some embodiments of the method, the ligand-regulated, stability-affecting fusion protein comprises the amino acid sequence identified by SEQ ID NO: 1. In some embodiments of the method, the ligand-regulated, stability-affecting fusion protein comprises the amino acid sequence identified by SEQ ID NO: 3.

Also disclosed herein is a “ligand-induced degradation” (LID) domain comprising SEQ ID No: 1. Further disclosed is a fusion protein comprising the LID domain.

Further disclosed herein is a cell comprising a nucleic acid encoding a fusion protein comprising a protein of interest fused in frame to a LID stability-affecting polypeptide. In some embodiments, the cell is stably transformed with the nucleic acid. In some embodiments, the stably transformed cell is implanted in a living animal. In some embodiments, the nucleic acid encodes a LID domain polypeptide comprising SEQ ID NO: 3.

In some embodiments, the system employs a ligand-responsive, stability-affecting polypeptide domain based on the estrogen receptor (ER) and one or more ER-ligands (agonists or antagonists). In some embodiments, the ER-ligand is selected from the group consisting of CMP8, 4-hydroxytamoxifen, fulvestrant and raloxifene.

The stability-affecting polypeptide domain can be used in the system disclosed herein to stabilize or destabilize a protein of interest (POI) depending on which ligand is used in the system. In some embodiments, the stability-affecting polypeptide domain acts as a degradation domain (DD)/ligand-induced stability domain, for example in the presence of estradiol as a ligand. In some embodiments, the stability-affecting polypeptide domain acts as a ligand-induced destabilization domain, for example in the presence of tamoxifen as a ligand.

In some embodiments, the nucleic acid sequence is in a viral vector. In some embodiments, the viral vector is a vaccinia virus.

In some embodiments of the system or method, introducing the nucleic acid comprises administering a viral vector comprising the nucleic acid to an animal in which the cell resides.

A related aspect of the methods and compositions are cells transfected with nucleic acids encoding a fusion protein comprising a protein of interest fused in frame to a stability-affecting protein. Expression of the fusion protein may be driven by the endogenous promoter, ideally reproducing the spatial and temporal expression patterns of the unmodified gene. The cells may be transfected, e.g., using an expression vector, or transduced (i.e., infected) using a viral vector, including but not limited to a vector derived from a retrovirus (e.g., a lentivirus), herpesvirus, pox virus, adenovirus, adenoassociated virus, or an RNA virus, such as poliovirus, flavivirus, alphavirus, or the like. The exemplary viral vector was based on a retrovirus.

The system was shown to be effective eukaryotic cells, including mammalian cells and protozoan parasites; therefore, the system can be expected to work in various eukaryotic cells, including those of humans, primates, rodents, dogs, cats, horses, cows, sheep, insects, amphibians, and parasites. The cells may be in culture or in a living organism. As noted above, the wild-type or naturally-occurring gene or allele encoding the POI may be deleted to facilitate study of the conditionally stabilized POI.

The present methods and compositions also allow the creation of transgenic animals harboring engineered alleles that direct the expression of a stability-affecting polypeptide fused to the POI. Expression of this fusion protein may be driven by the endogenous promoter, ideally reproducing the spatial and temporal expression patterns of the unmodified gene. The ability to specifically and conditionally stabilize a POI in a cell will enable the study of many proteins to determine their biological function and importance in a cell. The present methods and composition represent a significant improvement over current methods of conditional protein regulation.

The present disclosure provides a novel single stability-affecting protein domain capable of interacting with a cell-permeable, synthetic small-molecule ligand, where the presence, absence, or amount of the small-molecule ligand is used to modulate the stability of the fusion protein. The sequence encoding the stability-affecting protein domain is genetically fused to a coding sequence encoding a protein of interest to allow the rapid, reversible modulation of the fusion product in cells as well as in higher organisms. This technology provides the biological community with tools to directly interrogate protein function in living cells and animals with unprecedented control. This technology facilitates fundamental biological studies as well as more applied advances such as the creation of new and useful animal models of human diseases. With broad applicability, this technology is also useful in the pharmaceutical and biotech industries for validation of potential therapeutic targets.

### IV. Kits of Parts

The methods and compositions described herein may be packaged together with instructions for use, as in a kit of parts. Exemplary kits of parts include nucleic acids encoding stability-affecting proteins in-frame with a multiple cloning site and instructions for use of the kit. The instructions may contain information relating the inserting (i.e., “cloning”) a POI into a plasmid, in-frame with a stability-affecting protein. The instructions may also include dosing recommendations and hardware, such as syringes, to deliver the fusion protein to an organism or to cells.

### V. Examples

The following examples are illustrative in nature and are in no way intended to be limiting.

### Materials and Methods

### A. Cloning, Cell Culture, Transfections, and Transductions

Cell lines were cultured in DMEM supplemented with 10% heat-inactivated donor bovine serum (Invitrogen), 2 mM glutamine, 100 U/ml penicillin and 100 μg/ml streptomycin. All other cell lines were cultured with 10% heat-inactivated fetal bovine serum (Invitrogen), 2 mM glutamine, 100 U/ml penicillin and 100 μg/ml streptomycin.

The INX ecotropic packaging cell line was transfected using standard Lipofectamine 2000 protocols. Viral supernatants were harvested 48 h post-transfection, filtered. NIH3T3 cells were incubated with the retroviral supernatants supplemented with 4 μg/ml polybrene for 4 h at 37° C. Cells were washed once with PBS and cultured in growth media for 48 hrs to allow for viral integration, then assayed as described.

### B. Flow Cytometry

Twenty-four hours prior to analysis, transduced NIH3T3 cells were plated at 1×10⁵ cells per well of a 12-well plate. Cells were either treated with the ligand or mock treated with solvent only. Cells were removed from the plate using 0.02% trypsin—EDTA, and resuspended in 200 μL culture medium. Cells were analyzed at the Stanford Shared FACS Facility using Vantoo or LSRII with 10,000 events represented.

### C. Protein-of-Interest Origin, Immunoblotting and Antibodies

Proteins tested as fusions to LID domains were of the following origin: human replication protein A2, (RPA2, NM_{—}002946), human ubiquitin-conjugating enzyme (UBE2, BC108704), Mus musculus BCL2-associated X protein (Bax, NM_{—}007527), Mus musculus baculoviral IAP repeat-containing 5 (Birc5 or Survivin, NM_{—}009689), AcGFP, Aequorea coerulescens. All fusion proteins except AcGFP were epitope-tagged with human influenza hemagglutinin (HA). For immunoblots, the fusion proteins were detected with rat anti-HA monoclonal antibody (3F10, Roche) and AcGFP was detected with mouse monoclonal antibody anti-XFP antibody (JL-8, Clontech).

### Example I

### Ligand-Induced Destabilizing Domain

Human FKBP 2 protein was modified to include a 19-residue peptide extension at the C-terminus of the FKBP12 protein. Genetic diversity was engineered into this C-terminal extension using synthetic oligonucleotides and PCR, and this library of extended FKBP genes was fused to the 3′-end of the YFP gene. A library of plasmids was stably introduced into NIH3T3 cells using retrovirus, and FACS was used to screen the transduced cells for cells expressing YFP in the absence of the small molecule ligand Shield-1. When Shield-1 was added, YFP levels dropped significantly. Thus, upon addition of the small molecule ligand Shield-1, the Ligand-Induced Degradation (LID) domain and fusion protein with its N-terminal region were degraded by the proteosome machinery. Several rounds of screening, followed by single-cell-derived cloning revealed a single domain that displayed the desired behavior. This domain is identified herein by SEQ ID NO: 1.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A ligand-regulated conditional protein stability system, comprising:
a nucleic acid sequence encoding a fusion protein comprising a protein of interest fused in-frame to a stability-affecting protein domain, an optional spacer; and a degron; and
a ligand,
wherein, upon introduction of the nucleic acid sequence to a cell, the fusion protein is expressed, and wherein stability of fusion protein is modulated upon administration of the ligand.

2. The system of claim 1, wherein stability of fusion protein is increased upon administration of the ligand.

3. The system of claim 1, wherein stability of fusion protein is decreased upon administration of the ligand.

4. The system of claim 1, wherein the stability-affecting protein domain comprises SEQ ID NO: 1 or sequence having at least about 95% identity to SEQ ID NO: 1.

5. The system of claim 1, wherein the stability-affecting protein domain comprises SEQ ID NO: 3 or sequence having at least about 95% identity to SEQ ID NO: 3.

6. The system of claim 1, wherein the ligand is selected from the group consisting of CMP8, 4-hydroxytamoxifen, fulvestrant and raloxifene.

7. The system of claim 1, wherein the nucleic acid sequence is in a viral vector.

8. The system of claim 7, wherein the viral vector is a vaccinia virus.

9. A fusion protein comprising SEQ ID NO: 1 or sequence having at least about 95% identity to SEQ ID NO: 1.

10. A fusion protein comprising SEQ ID NO: 3 or sequence having at least about 95% identity to SEQ ID NO: 3.

11. A method for conditionally stabilizing a protein of interest, comprising:
fusing a nucleic acid encoding the protein of interest in-frame to a nucleic acid encoding a stability-affecting protein domain to produce a nucleic acid encoding a fusion protein;
introducing the nucleic acid encoding the fusion protein into a cell;
expressing the fusion protein in the cell; and
exposing the cell to a ligand to modulate stability of the fusion protein.

12. The method of claim 11, wherein the domain comprises an amino acid sequence identified by SEQ ID NO: 1 or an amino acid sequence having at least about 95% identity to SEQ ID NO: 1.

13. The method of claim 11, wherein the domain comprises an amino acid sequence identified by comprises SEQ ID NO: 3 or an amino acid sequence having at least about 95% identity to SEQ ID NO: 3.

14. The method of claim 11, wherein the cell is stably transformed with the nucleic acid.

15. The method of claim 14, wherein the stably transformed cell is implanted in a living animal.

16. The method of claim 11, wherein the nucleic acid sequence is in a viral vector.

17. The method of claim 16, wherein the viral vector is a vaccinia virus.

18. The method of claim 11, wherein the ligand is selected from the group consisting of CMP8, 4-hydroxytamoxifen, fulvestrant and raloxifene.

19. The method of claim 11, wherein introducing the nucleic acid comprises administering a viral vector comprising the nucleic acid to an animal in which the cell resides.

20. A cell comprising a nucleic acid encoding a fusion protein comprising a protein of interest fused in frame to a ligand-regulated, stability-affecting polypeptide.

21. The cell of claim 20, wherein the cell is stably transformed with the nucleic acid.

22. The cell of claim 21, wherein the stably transformed cell is implanted in a living animal.

23. A kit of parts comprising the system of claim 1 along with instructions for use.
